(19) United States
(12) Patent Application Publication
(10) Pub. No.: US 2003/0036517 A1
(43) Pub. Date: Feb. 20, 2003

(54) NUCLEIC ACID MEDIATED RNA TAGGING AND RNA REVISION

(76) Inventors: BRUCE A. SULLENGER, DURHAM, NC (US); NING LAN, DURHAM, NC (US); SEONG-WOOK LEE, SEOUL (KR); LYNN MILICH, DURHAM, NC (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/205,520

(22) Filed: Dec. 3, 1998

Related U.S. Application Data
(60) Provisional application No. 60/067,834, filed on Dec. 5, 1997.

(51) Int. Cl. A61K 48/00; C07H 21/02

(52) U.S. Cl. 514/44; 536/23.1

(57) ABSTRACT

A method of identifying accessible regions in a target RNA molecule using trans-splicing nucleic acid molecules is disclosed. Also disclosed is a method of revising mutant globin gene sequences using trans-splicing nucleic acid molecules and a method of tagging nucleic acid molecules with Tag moieties using trans-splicing nucleic acid molecules.

### CROSS-REFERENCE TO RELATED APPLICATIONS

This application claims priority to U.S. Provisional Application 60/067,834, Bruce A. Sullenger, et al., entitled “NUCLEIC ACID MEDIATED RNA TAGGING AND RNA REVISION”, filed Dec. 5, 1997, which is hereby incorporated herein by reference in its entirety, including any drawings and figures.

This invention was made with Government support under Grant No(s) HL57606 awarded by the National Institutes of Health. The Government has certain rights in the invention.

### BACKGROUND OF THE INVENTION

This invention relates to method and reagent for tagging nucleic acid molecules and repairing RNA molecules.

The following is a brief description of RNA splicing and RNA processing reactions. This summary is not meant to be complete but is provided only for understanding of the invention that follows. This summary is not an admission that all of the work described below is prior art to the claimed invention.

During gene expression, the information contained in a given protein encoding gene is directly copied into the corresponding pre-messenger RNA by transcription. The information embedded in this RNA is not fixed however and can be modified by splicing (Ruby et al., 1991 TIGS 7,79; Guthrie, 1991, Science, 253, 157) or editing (Sollner-Webb, Curr. Opin. Cell Bio. 3, 1056) to remove, add or rewrite parts of the initial transcript. The self-splicing reaction of the group I intron ribozyme from Tetrahymena thermophila is perhaps the most thoroughly understood reaction that revises RNA. The intron performs two consecutive transesterification reactions to liberate itself and join flanking exon sequences (FIG. 1A) (Been et al., 1986, Cell, 47, 207). Careful analysis of this self-splicing reaction over the past decade has illustrated that the vast majority of sequence requirements for such excision are contained within the intron. No specific sequence requirements exist for the 3′ exon, and the only specific sequence requirement for 5′ exons is to have a uridine (U) preceding the cleavage site. In addition, base pairing must be maintained between the end of the 5′ exon and the 5′ exon-binding site present in the ribozyme so that the ribozyme can hold onto the 5′ exon after cleavage. These base pairs can be composed of any sets of complementary nucleotides however.

Ribozyme mediated trans-splicing. In addition to performing self-splicing, the group I ribozyme from Tetrahymena can trans-splice an exon attached to its 3′ end onto a separate 5′ exon RNA (FIG. 1B). In this reaction, the 5′ exon is not covalently attached to the ribozyme but is bound via base pairing through the 5′ exon binding site on the ribozyme. In the process of pairing, a U is positioned across from the guanosine present at the 5′ end of the 5′ exon binding site. Once positioned, the ribozyme cleaves the bound substrate RNA at the reconstructed 5′ splice site and ligates its 3′ exon onto the 5′ exon cleavage product (FIG. 1B). Trans-splicing by group I ribozymes is extremely malleable. Virtually any U residue in a 5′ exon can be targeted for splicing by altering the nucleotide composition of the 5′ exon binding site on the ribozyme to make it complementary to a target sequence present on the substrate RNA. Because no specific 3′ exon sequences are required, virtually any 3′ exon sequence can be spliced onto a targeted U residue by such a reaction.

Directed RNA revision by trans-splicing in bacteria. A trans-splicing ribozyme can be employed to revise the sequence of targeted RNAs. In the first example of this application, we recently demonstrated that a trans-splicing group I ribozyme from Tetrahymena can be employed to repair truncated lacZ transcripts (Sullenger et al., 1994, Nature 371, 619; Sullenger et al., U.S. Pat. No. 5,667,969; both are incorporated by reference herein). In this system, a 3′ exon sequence encoding the restorative lacZ sequence was attached to the splicing ribozyme. For trans-splicing to correct the defective lacZ messages, the ribozyme must recognize the truncated 5′ lacZ transcript by base pairing, cleave off additional nucleotides, hold onto the 5′ lacZ cleavage product, and ligate the restorative lacZ 3′ exon sequence onto the cleaved 5′ product to yield the proper open reading frame for translation. It was shown that the ribozyme could faithfully accomplish such RNA revision both in vitro and in Escherchia coli. Furthermore, in E. coli the repaired RNAs went on to be translated to produce a functional enzyme.

Inoue et al., 1985, Cell 43, 431; state that short oligonucleotides of 2-6 nucleotides can undergo intermolecular exon ligation or splicing in trans. It indicates that “long 5′ exons should be reactive provided that three conditions are met: the exon must have a 3′ hydroxyl group, it must terminate in a sequence similar to that of the 3′ end of the 5′ exon, and the 3′ terminal sequence must be available as opposed to being tied up in some secondary structure. Thus, it appears that exon switching is possible in this system, though limited by the availability of alternative 5′ exons that meet the above criteria. These could include transcripts that are not 5′ exons from other precursors, since RNA polymerases always leave 3′ hydroxyl ends.”

Haseloff et al., U.S. Pat. No. 5,641,673 describe a method of “cell ablation . . . that provides a toxic product to a host cell in vivo in a targetted, regulated manner utilizing group I trans-splicing ribozyme.”

Mitchell et al., International PCT Publication No. WO 97/22250, describe a method of trans-splicing to “selectively kill target cells.”

### SUMMARY OF THE INVENTION

This invention features a method in which a mutant beta-globin transcripts are altered by use of a splicing reaction in vivo or in vitro. It involves the manipulation of genetic information to ensure that a useful transcript is provided within a cellular system or extract. The invention also features a method of identifying regions in a target RNA that are accessible to interaction with separate macromolecules such as nucleic acid molecules, using trans-splicing nucleic acid molecules. The invention further describes a method of attaching non-nucleic acid Tags to target nucleic acid molecules.

In a preferred embodiment, the trans-splicing nucleic acid molecules are enzymatic nucleic acid molecules. More specifically the trans-splicing nucleic acid molecules are derived from group I (Sullenger et al., supra) or group II introns (Jacquier, 1990, TIBS 15, 351; Michels et al., 1995, Biochemistry, 34, 2965; Chanfreau et al., 1994, Science, 266, 1383; Mueller et al., 1993, Science, 261, 1035; Jarrell et al., U.S. Pat. No. 5,498,531; all are incorporated herein by reference).

In another preferred embodiment, the trans-splicing nucleic acid molecules facilitate trans-splicing reaction in the presence of one or more cellular factors, such as protein factors (Bruzik et al., supra; Jarrell supra; Ghetti et al., 1995, Proc. Natl. Acad. Sci., 92, 11461; all are incorporated by reference herein). Preferably, such trans-splicing nucleic acid molecules are derived from pre-messenger RNA introns, but can also be derived from other introns such as group I and group II.

In a first aspect the invention features a method of replacing a region of a mutant beta-globin RNA molecule containing one or more mutations with a desired beta-globin sequence using trans-splicing nucleic acid molecules, to generate a beta-globin RNA molecule able to express a protein with normal beta-globin protein attributes (FIG. 2A). The method involves: a) contacting the target RNA molecule (e.g., mutant beta-globin RNA) in vitro or in vivo with a trans-splicing nucleic acid molecule (e.g. group I intron ribozyme, group II intron ribozyme, pre-mRNA intron or the like), comprising a Tag with a defined sequence (e.g., desired beta-globin sequence with out deleterious mutations); b) the trans-splicing nucleic acid molecule is incubated with the target RNA molecule under conditions suitable for trans-splicing reaction to occur; the trans-splicing reaction removes the defective (mutant) region of the beta-globin RNA and in its place covalently attaches the desired beta-globin sequence in the target RNA molecule.

By “desired beta-globin” sequence is meant sequence of beta-globin RNA that does not have mutations that are deleterious to the normal function of a wild type beta-globin protein (see Andrin et al., 1994, Biochem. Cell. Bio. 72, 377; Orkin, 1990, Cell, 63, 665).

By “normal beta-globin protein attributes” is meant function or properties of a beta-globin protein that are not associated with a disease or a condition (see Andrin et al., 1994, Biochem. Cell. Bio. 72, 377; Orkin, 1990, Cell, 63, 665).

In a second preferred embodiment, the invention features a method of converting mutant beta-globin RNA molecule containing one or more mutations into a chimeric beta-gamma-globin sequence using trans-splicing nucleic acid molecules, to generate a RNA molecule able to express a protein with normal gamma-globin protein fuction and properties. The method involves: a) contacting the target RNA molecule (e.g., mutant beta-globin RNA) in vitro or in vivo with a trans-splicing nucleic acid molecule (e.g. group I intron ribozyme, group II intron ribozyme, pre-mRNA intron or the like), comprising a Tag with a defined sequence (e.g., a gamm-globin sequence); b) the trans-splicing nucleic acid molecule is incubated with the target RNA molecule under conditions suitable for trans-splicing reaction to occur; the trans-splicing reaction removes the defective (mutant) region of the beta-globin RNA and in its place covalently attaches the gamma-globin sequence in the target RNA molecule to generate a chimeric beta-gamma-globin RNA.

By “chimeric beta-gamma-globin sequence” is meant a gamma-globin sequence having one or more regions of beta-globin RNA and where the chimeric sequence is able to express a protein having the function and one or more properties of a gamma globin protein (see Andrin et al., 1994, Biochem. Cell. Bio. 72, 377; Orkin, 1990, Cell, 63, 665).

The trans-splicing nucleic acid molecule is not naturally associated with the Tag sequence since it is not generally desired to splice the Tag sequence of a naturally occurring nucleic acid molecule with a target RNA molecule. Rather, the Tag sequence is chosen or selected to have a desired function once spliced with the target nucleic acid molecule.

In preferred embodiments, the catalytic nucleic acid molecule is able to cleave and splice, e.g., it has a group I (Sullenger et al., supra) or group II intron (Jacquier, 1990, TIBS 15, 351; Michels et al., 1995, Biochemistry, 34, 2965; Chanfreau et al., 1994, Science, 266, 1383; Mueller et al., 1993, Science, 261, 1035; Jarrell et al., U.S. Pat. No. 5,498,531; all are incorporated herein by reference) motif; the method is performed in vitro or in vivo with an RNA target; and the method can be used to treat genetic disease in a gene therapy type manner, for example, by correcting an abnormal transcript.

In other aspects, the invention features catalytic nucleic acid molecules having a desired Tag sequence as a 3′ exon encoding at least a portion of a useful gene which can be used in gene therapy. The Tag sequence can also be attached to a target RNA (for example associated with a certain disease condition) in a biological sample, for example from a patient for diagnostic purposes; the Tag sequence is used as an indicator of the presence and quantity of the target RNA in a sample. Such a molecule can be spliced with and thereby correct or modify the expression of other target RNA molecules. The invention also features vectors encoding such catalytic nucleic acid molecules.

In a third preferred embodiment, the invention features a method of identifying a region or regions in a target RNA molecule that is accessible to interaction (e.g., hybridization) with a separate nucleic acid molecule involving: a) contacting the target RNA molecule in vitro or in vivo with an enzymatic nucleic acid molecule with trans-splicing activity (e.g. group I intron ribozyme, group II intron ribozyme or the like), comprising a Tag with a defined sequence; b) the enzymatic nucleic acid molecule is incubated with the target RNA molecule under conditions suitable for trans-splicing reaction to occur; the trans-splicing reaction covalently attaches the Tag sequence to the target RNA molecule to form a chimeric RNA molecule; and c) identifying the accessible region in the target RNA; the region of target RNA molecule where the Tag sequence has been inserted (accessible region) is readily identified using standard molecular biology techniques such as reverse transcription and polymerase chain reaction.

In a further embodiment, the invention features a method of identifying a region or regions in a target RNA molecule that is accessible to interaction (e.g., hybridization) with a separate nucleic acid molecule including the step of contacting the target RNA molecule in vitro or in vivo with an enzymatic nucleic acid molecule with trans-splicing activity (e.g. group I intron ribozyme, group II intron ribozyme or the like), comprising a Tag with a defined sequence. The enzymatic nucleic acid molecule includes a target binding domain and an enzymatic domain, where the target binding domain has a randomized region. The enzymatic nucleic acid molecule with randomized binding arm is contacted with target RNA molecule under conditions suitable for the attachment of the Tag sequence to the target RNA. The region of the target RNA with the inserted Tag sequence is identified readily using standard molecular biology techniques.

By “randomized region” is meant a region of completely random sequence and/or partially random sequence. By completely random sequence is meant a sequence wherein theoretically there is equal representation of A, U, G and C nucleotides or modified derivatives thereof, at each position in the sequence. By partially random sequence is meant a sequence wherein there is an unequal representation of A, U, G and C nucleotides or modified derivatives thereof, at each position in the sequence. A partially random sequence can therefore have one or more positions of complete randomness and one or more positions with defined nucleotides.

By “separate nucleic acid molecule” is meant a nucleic acid molecule capable of interacting with a target nucleic acid molecule and modulate the expression and/or function of the target nucleic acid molecule. Such separate nucleic acid molecules include enzymatic nucleic acid molecules, antisense oligonucleotides, triplex forming oligonucleotides, peptide nucleic acid molecules, aptamers, 2-5 A antisense chimeras, and others.

By “antisense oligonucleotide” it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review see Stein and Cheng, 1993 Science 261, 1004; Agrawal et al., U.S. Pat. No. 5,591,721; Agrawal, U.S. Pat. No. 5,652,356).

By “2-5 A antisense chimera” it is meant, an antisense oligonucleotide containing a 5′ phosphorylated 2′-5′-linked adenylate residues. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300).

By “triplex forming oligonucleotides (TFO)” it is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504).

By “oligonucleotide” as used herein is meant a molecule having two or more nucleotides. The polynucleotide can be single, double or multiple stranded and may have modified or unmodified nucleotides or non-nucleotides or various mixtures and combinations thereof.

By “nucleic acid molecule” as used herein is meant a molecule having nucleotides. The nucleic acid can be single, double or multiple stranded and may comprise modified or unmodified nucleotides or non-nucleotides or various mixtures and combinations thereof. An example of a nucleic acid molecule according to the invention is a gene which encodes for macromolecule such as a protein.

By “complementarity” as used herein is meant a nucleic acid that can form hydrogen bond(s) with other nucleic acid sequence by either traditional Watson-Crick or other non-traditional types (for example, Hoogsteen type) of base-paired interactions.

By “enzymatic nucleic acid” it is meant a nucleic acid molecule capable of catalyzing reactions including, but not limited to, site-specific cleavage and/or ligation of other nucleic acid molecules, cleavage of peptide and amide bonds, and trans-splicing (see for example (Zaug et al., 324, Nature 429 1986; Cech, 260 JAMA 3030, 1988; Usman & McSwiggen, 1995 Ann. Rep. Med. Chem. 30, 285-294; Christoffersen and Marr, 1995 J. Med. Chem. 38, 2023-2037; all are incorporated by reference herein). Such a molecule with endonuclease activity may have complementarity in a substrate binding region to a specified gene target, and also has an enzymatic activity that specifically cleaves RNA or DNA in that target. This complementarity functions to allow sufficient hybridization of the enzymatic RNA molecule to the target RNA or DNA to allow the cleavage to occur. 100% complementarity is preferred, but complementarity as low as 50-75% may also be useful in this invention. The nucleic acids may be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving/ligation activity to the molecule.

By “enzymatic portion” or “catalytic domain” is meant that portion/region of the ribozyme necessary for catalytic activity (for example see FIG. 1).

By “substrate binding arm” or “substrate binding domain” is meant that portion/region of a ribozyme which is complementary to (i e., able to base-pair with) a portion of its substrate. Generally, such complementarity is 100%, but can be less if desired. For example, as few as 10 bases out of 14 may be base-paired. Such arms are shown generally in FIG. 1 and 2. That is, these arms contain sequences within a ribozyme which are intended to bring ribozyme and target RNA together through complementary base-pairing interactions. The ribozyme of the invention may have binding arms that are contiguous or non-contiguous and may be of varying lengths. The length of the binding arm(s) are preferably greater than or equal to four nucleotides; specifically 12-100 nucleotides; more specifically 14-24 nucleotides long. If two binding arms are chosen, the design is such that the length of the binding arms are symmetrical (i e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like). Those of ordinary skill in the art will recognize that other motifs than those of the group I and group II introns may also be manipulated to provide useful trans-splicing activity.

The conditions chosen for the contacting step and the trans-splicing step may be those naturally occurring within a cell, or may be manipulated in vitro to ensure that the splicing reaction will occur. These conditions are well known to those in the art, for example, as described by Inoue et al., supra.

In a fourth embodiment the invention features a method of attaching a Tag moiety other than nucleic acid to a target nucleic acid using enzymatic trans-splicing nucleic acid molecules, comprising the step of contacting the target nucleic acid molecule with the enzymatic trans-splicing nucleic acid molecule comprising a Tag under conditions suitable for the attachment of the Tag.

By “Tag sequence” is meant a non-naturally occuring sequence with a few nucleotides (10-500 nucleotides) or may be significantly greater and may represent almost all of a molecule encoding a gene product (i.e., at least 1 to 5 kbases).

By “Tag” is meant a chemical moiety that can be linked to a target nucleic acid molecule using a trans-splicing nucleic acid molecule. Non-limiting examples of a Tag are nucleic acid, nucleotides, nucleoside triphosphate, lipid moiety, carbohydrate moiety, biotin, a detergent, peptide, aminoacid, antibiotic, and others. More specifically the Tag moiety is selected from a group consisiting of a lipid, carbohydrate, vitamin, biotin, a fluoroscence compound (e.g., fluorescein, rhodamine and the like), peptide (e.g., peptides to facilitate intracellular trafficking of nucleic acid molecules), aminoacid, antibody and an antibiotic.

By “target nucleic acid molecule” is meant any nucleic acid molecule that serve as a target for interaction with a trans-splicing nucleic acid molecule.

The “chimeric RNA molecule” is one which is a non-naturally occuring not present in the system prior to the initiation and completion of trans-splicing reaction. Alternatively, it may be a completely novel structure which does not occur in nature, but which is useful in gene therapeutic treatment of an organism.

Other features and advantages of the invention will be apparent from the following description of the preferred embodiments thereof, and from the claims.

### DESCRIPTION OF THE PREFERRED EMBODIMENTS

The drawings will briefly be described.

### DRAWINGS

FIG. 1A is a diagrammatic representation showing splicing reactions of the group I intron from Tetrahymena. FIG. 1B, shows a schemmatic representation of a strategy for targeted trans-splicing.

FIG. 2 shows a scheme for ribozyme-mediated repair of sickle beta-globin transcripts. A) Scheme for the conversion of β^{S}-globin mRNAs into transcripts encoding desired β-globin. b) Scheme for the conversion of β^{S}-globin mRNAs into transcripts encoding chimeric β-γ-globin. Xₘ, sickle beta-globin point mutation; γ-3′exon, restorative globin sequence.

FIG. 3 shows a scheme for identifying accessible regions within beta-globin RNA using trans-splicing ribozyme. a) A library of trans-splicing ribozymes containing randomized guide sequences (5′-GNNNNN-3′) is incubated with beta-globin mRNA. Ribozymes in the library that react with accessible uridine residues on beta-globin transcripts attach their 3′ exon Tag to these sites. These “Tagged” reaction products are amplified by RT/PCR using a primer (D) specific to the 3′exon Tag and a primer (U) specific for the beta-globin RNA. These amplified fragments are cloned and sequenced to determine which uridines are present at the ribozyme reaction sites. b) Mapping results. Nucleotide positions are presented for the accessible uridines identified from in vitro (left) and in vivo (right) mapping analysis. The number of individual clones containing a given uridine at the splice site is indicated. Position 70 denotes the nucleotide that is altered in sickle beta-globin transcripts.

FIG. 4. Trans-splicing a 3′ exon Tag onto beta-globin transcripts. a) RT-PCR analysis of trans-spliced RNA products generated in vitro. Active (Rib61-3′tag) and inactive (Rib61d-3′tag) ribozymes were incubated with a truncated β^{S}-globin transcript (β^{S}-61) that contains the first 61 nucleotides of the RNA, the full length β^{S}-globin transcript (β^{S}-FL) or total RNA isolated from erythrocyte precursors derived from normal umbilical cord blood (UCB RNA) or from peripheral blood of sickle cell patients (SC RNA). Amplification of trans-splicing reaction products is expected to yield a 93 base pair product. A 50 base pair DNA ladder provided molecular mass markers. b) RT-PCR analysis of trans-spliced RNA products generated in RBC precursors. Erythrocyte precursors derived from normal umbilical cord blood (UCB) or peripheral blood of sickle cell patients (SC) were mock transfected (mock and mix) or transfected with the active (Rib61-3′tag) or inactive (Rib61d-3′tag) ribozymes. RNA was harvested from these cells and trans-spliced products analyzed as in FIG. 4a. In the “mix” sample, Rib61-3′tag was added to the RNA extraction buffer prior to RNA isolation.

FIG. 5. Converting β^{S}-globin transcripts into γ-globin encoding RNAs. a) In vitro trans-splicing reaction. Body-labeled ribozyme-3′γ globin RNA (Rib61-3′γ) was incubated under splicing conditions with full length β^{S}-globin RNA (β^{S}-FL), truncated β^{S}-globin RNA (β^{S}-61A₃:61 nucleotides of β^{S}-globin RNA plus 3 adenosine residues) and a 13 nucleotide substrate (5′SA₅:GGGCACCUAAA) for the indicated times. Trans-spliced products (5′S-3′g and β^{S}-61-3′g) and free ribozyme (Rib61) are indicated. b) RT-PCR analysis of amended RNAs generated in erythrocyte precursors from normal umbilical cord blood (UCB) or peripheral blood of sickle cell patients (SC). Cells were mock transfected (mock and mix) or transfected with the active (Rib61-3′γ) or inactive (Rib61d-3′γ) ribozymes. Amended RNAs were amplified by RT/PCR and yield a DNA fragment of 62 base pairs. In the mix samples, Rib61-3′γ was added to the RNA extraction buffer before RNA isolation. Molecular weight marker of 72 and 50 base pairs are shown. c) Sequence of amended β^{S}-globin transcripts. The expected sequence for a corrected transcript around the splicing junction is shown, with the complement to the IGS shaded and the uridine at position 61 circled. β^{S}-globin and γ-globin sequences are provided for comparison and the mutant nucleotide in the sickle β-globin transcript indicated. Translation of the amended transcript would yield γ-globin with three amino acids derived from β-globin.

### TARGET SITE ACCESSIBILITY

To successfullly employ separate nucleic acid molecules such as, antisense, ribozyme or other nucleic acid-based molecules, to modulate gene expression, these oligonucleotides must be able to interact with their intended target nucleic acid (e.g., RNA) inside cells. Unfortunately, cellular RNAs are not linear but rather adopt highly folded structures that make most of the nucleotides on the target RNA inaccessible to nucleic acid molecules, such as antisense and ribozyme molecules. Thus, it has become useful in the field of nucleic acid therapeutics to determine which regions of folded target RNAs are accessible to interactions (e.g., base pairing) with these separate nucleic acid molecules. To identify such accessible regions, the field has made a great number of different antisense or ribozyme molecules for a given target RNA and assessed the activity of the individual molecules in solution in a test tube (see for example McSwiggen et al., U.S. Pat. No. 5,525,468).

Applicant has developed a novel approach to determine which regions of target RNAs are accessible by using a library of trans-splicing ribozymes. This approach takes advantage of the fact that such trans-splicing ribozymes covalently attach nucleotide Tag sequences onto their reaction sites. These attached Tag sequences serve as a convenient molecular tag that then allows one to subsequently identify the reaction site after simple reverse transcription (RT) and polymerase chain reaction (PCR) amplification (RT/PCR) and sequencing. Because this tagging can proceed in cells or in cell free systems using total RNA isolated from cells, it offers several significant advantages over the currently employed technology. First, it allows one to simultaneously map a number of target transcripts by simply employing different amplification primers which greatly increases mapping through put. Second, it allows one to map the actual target RNA of interest inside cells by transfecting the library of trans-splicing ribozymes directly into cells. Thus, this novel approach will greatly expedite the identification of accessible regions of target RNAs and therefore will be of great value in the development of useful nucleic acid-based therapeutic agents.

It should be noted that other approaches for amplification of reaction products exist including amplification of chimeric RNAs by Q beta replicase. The RNA Tag can be made to contain the substrate recognition sequence for Q beta replicase which will be transferred to the target RNA during the reaction. To generate more reaction/amplification specificity, part of the sequence required to generate the Q beta replicase substrate RNA can be made to be part of the substrate RNA upstream of the reaction site. Thus the Q beta substrate RNA will only be generated by tagging of a specified reaction site with the appropriate RNA sequence. This strategy can be employed to make the reaction/amplification more specific when using RT/PCR amplification as well.

It should be noted that RNA tagging can be made to proceed in cells as well as in the test tube. Therefore, tagging and even amplification (for example if Q beta replicase is coexpressed inside the cells) can be performed in a living cell providing a novel approach to nucleic acid amplification and diagnostics.

Attachment of Molecular Tag:

As previously described Applicant provides a means of attaching molecular tags to targeted nucleic acids using trans-splicing reaction. The Tag can be a nucleic acid sequence (Tag sequence). The molecular tags and targets however do not necessarily have to be composed of nucleic acids. For example, in vitro selection has allowed various groups to generate enzymatic nucleic acid molecules that can react with a range of non-nucleic acid molecules (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Robertson et al., 1997, Curr. Bio. 7, R376; Jhaveri et al., 1997, Ann. Rep. Comb. Chem. Mol Diversity, 1, 169; all are incorporated by reference herein). Thus trans-splicing forms of these ribozymes should be able to covalently attach molecular tags to the non-nucleic acid reaction sites. Thus ribozymes can be employed to specifically modify a variety of substrate molecules by covalently attaching molecular tags to their targets.

Similarly, the molecular Tags do not have to be composed of nucleic acid sequence. One can simply alter the molecular composition of the 3′ exon Tag attached to the ribozyme. Just as in vitro selection has allowed for the generation of novel ribozymes with new cleavage activities, similar selection should allow for the development of ribozymes that can covalently attach novel Tags to target molecules. Thus ribozymes can be developed that can covalently modify a range of target molecules in a variety of ways. Such ribozymes can be used for a number of diagnostics and in manufacturing applications. For example, if one wants to a make a soap that has a specific type of chemical linkage that is difficult to generate by classical organic chemistry techniques, now one can consider the generation of a ribozyme that will recognize the precursor of the final soap product, say a certain lipid, catalytically react with the lipid and covalently transfer a molecular group (the Tag in this case) to the target lipid to modify it in the desired manner. Thus, the ability of ribozymes to covalently attach molecules to specific substrate molecules allows us to employ ribozyme in ways that were not previously envisioned.

Conventional gene therapy. In this era of molecular medicine, the genetic basis for an increasing number of inherited diseases including many types of cancer is being discovered. Gene therapy represents a new and exciting approach for the treatment of such diseases (Morgan, 1993, Ann. Rev. Biochem., 62:191-217). In its conception, gene therapy seemed quite simple. To treat a genetic deficiency give a functional copy of the defective gene to the cells of the deficient patient. To accomplish this in practice, most often a viral vector is used to transfer a cDNA copy of the wild type gene, which is usually under the control of a heterologous promoter, to cells harboring a mutant version. If the human genome were a simple warehouse of information, this approach would be quite successful. Unfortunately for the gene therapist, our genome appears to be extremely complicated, and expression of the information contained within it is apparently highly regulated. This complexity may severely limit the utility of the simple gene “add back” approach to gene therapy. Regrettably, cDNA versions of genes that are integrated in incorrect locations in the genome and that are expressed from heterologous promoters will almost assuredly not recapitulate the normal expression pattern of their endogenous counterparts. Therefore, significant technical advances may be necessary if the simple gene add back approach is to become useful for the treatment of genetic disorders associated with genes which require regulated expression to function properly.

Because genes associated with tumorigenesis normally control cell growth and differentiation, their expression will most likely have to be tightly regulated to coordinate cell cycle progression and development. At least three observations support this theory. First, deregulated expression of the transcription factor E2F-1 engenders premature entry into S-phase and can lead to p53 dependent apoptosis suggesting that expression of cell cycle proteins must be properly regulated for normal cell replication to proceed (Wu et al., 1994 Proc. Natl. Acad. Sci. USA, 91:3602-3606; Qin et al., 1994 Proc. Natl. Acad. Sci. USA, 91:10918-10922; Shan et al., 1994 Mol. Cell. Biol., 14:8166-8173; Kowalik et al., 1995, J Virol 69:2491-2500). Furthermore, as pointed out in a recent review by Weinberg (Cancer Surveys 1992;12:43-57), it has been difficult to restore normal growth to tumor cells harboring a mutant Rb (retinoblastoma) gene by retroviral vector mediated-transfer of a wild type cDNA copy of Rb to such cells. Although some groups have reported success with such experiments other have found that introduction of the Rb gene profoundly inhibited cell growth making it difficult to generate enough cells to perform experiments. Such results led Weinberg to state: “Perhaps a well regulated cloned Rb gene rather than one driven by a strong constitutive transcriptional promoter will yield cells that have lost tumorigenisity without loss of in vitro growth potential” (supra). If such regulated expression is required to correctly revert tumorigenisity in vitro, it will almost certainly be useful for reversion in vivo. Finally, it has been demonstrated that overexpression of the wild type p53 gene aberrantly alters growth and differentiation of normal human keratinocytes eventhough these cells normally express some natural level of the p53 protein from their wild type, endogenous genes (Woodworth et al., Cell Growth & Differ 1993;4:367-376). In these experiments, the extra p53 gene was introduced into primary keratinocytes using a retroviral vector, and this p53 cDNA version of the gene was expressed from the Moloney murine leukemia virus LTR promoter. The cells transduced with the p53 gene were shown to express only a modest 2-4 fold increase in the wild type p53 protein as compared to cells transduced with control vectors. This modest additional p53 expression however resulted in extremely reduced growth rate, altered morphological differentiation and aberrant expression of genes normally associated with correct differentiation of keratinocytes. Thus coordinated expression of the p53 gene is apparently important for proper growth, development and differentiation of primary human cells and incorrect expression can lead to dramatic phenotypic aberrations.Tumor suppressor genes are often mutated in transformed cells. Thus loss of tumor suppressor function appears to be a critical event during neoplastic transformation. In fact, about 60% of human cancers have mutations in the p53 tumor suppressor gene suggesting that p53 mutations may be the most common events in neoplastic transformation. A variety of mutations can apparently inactivate the p53 protein. Some cells have totally lost the p53 gene, however most express mutant p53 transcripts that contain missense point mutations between codons 120 and 290 of the 393 codon long gene. In breast cancers, the p53 gene is mutated approximately 40% of the time with most mutations found in exons 5-8 of the gene. Therefore, trans-splicing nucleic acid molecules can be used to repair the mutant p53 transcripts present in various tumor cells to restore the regulated expression of p53 and revert such cells from their transformed phenotypes.

Trans-Splicinz Ribozymes:

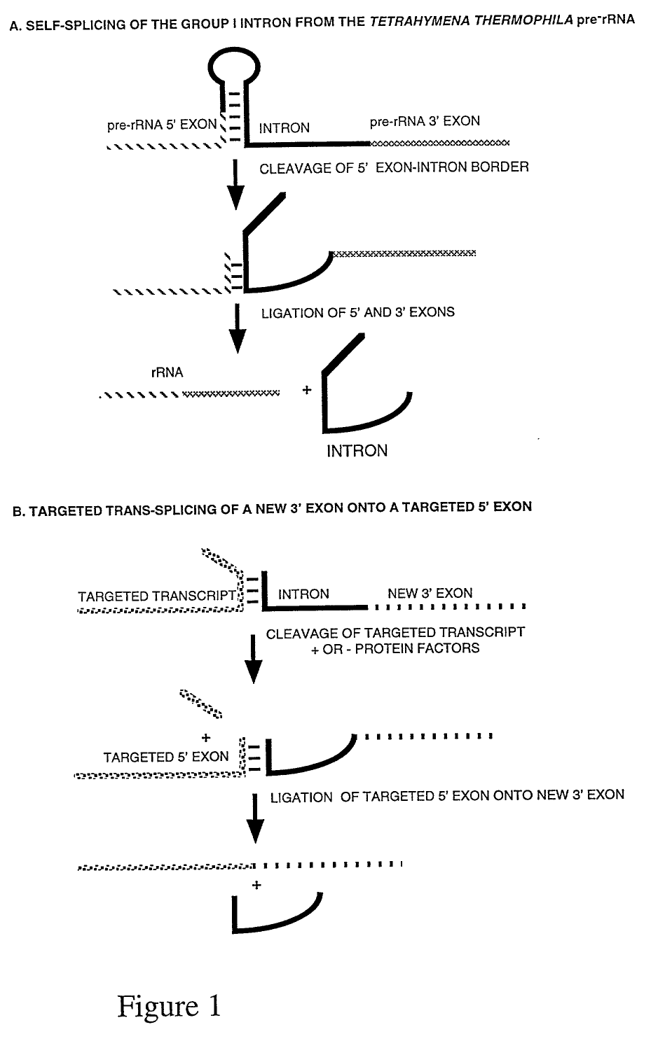

The general scheme for a targeted trans-splicing is shown in FIG. 1. Those in the art will recognize that any enzymatic nucleic acid molecule having the appropriate splicing activity can be used in the invention. The trans-splicing ribozymes are those that are known in the art (for e.g., group I or group II derived) or can be enzymatic nucleic acid molecules selected and/or evolved using selection techniques known in the art. There are several reports on in vitro selection protocols; following are examples of publications relating to the in vitro selection techniques all of which are incorporated herein by reference-Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Robertson et al., 1997, Curr. Bio. 7, R376; Jhaveri et al., 1997, Ann. Rep. Comb. Chem. Mol Diversity, 1, 169).

Alternatively, as discussed above, these molecules can be supplemented by other molecules having a suitable splicing activity, or by spliceosomes or splicing factors. The various splicing factors and spliceosomes are well known in the art, and this activity is generally described by Bruziket al., 1992, Nature 360, 692, hereby incorporated by reference herein. The invention concerns splicing of target nucleic acid molecules and Tag sequence which are not normally spliced together within a cell as described by Bruzik et al., supra. Rather, as described above, a Tag sequence is selected such that a useful function can be achieved in a gene therapeutic fashion.

Generally, the reaction involves base pairing of the catalytic nucleic acid molecule with the targeted transcript, cleavage of the targeted transcript, and then ligation of the3′ exon (Tag sequence) with this targeted 5′ exon. The catalytic nucleic acid is removed in the reaction. As will be noted, the specificity of the reaction can be changed by alteration of the substrate binding site in the catalytic nucleic acid molecule by methods well known in the art.

Optimizing Ribozyme Activity:

Catalytic activity of the ribozymes described in the instant invention can be optimized as known in the art. The details will not be repeated here, but include altering the length of the ribozyme binding arms, or chemically synthesizing ribozymes with modifications (base, sugar and/or phosphate) that prevent their degradation by serum ribonucleases and/or enhance their enzymatic activity (see e.g., Eckstein et al., International Publication No. WO 92/07065; Perrault et al., 1990 Nature 344, 565; Pieken et al., 1991 Science 253, 314; Usman and Cedergren, 1992 Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of enzymatic RNA molecules). Modifications which enhance their efficacy in cells, and removal of bases from stem loop structures to shorten RNA synthesis times and reduce chemical requirements are desired. (All these publications are hereby incorporated by reference herein).

There are several examples in the art describing sugar and phosphate modifications that can be introduced into enzymatic nucleic acid molecules without significantly effecting catalysis and with significant enhancement in their nuclease stability and efficacy. Ribozymes are modified to enhance stability and/or enhance catalytic activity by modification with nuclease resistant groups, for example, 2′-amino, 2′-C-allyl, 2′-flouro, 2′-O-methyl, 2′-H, nucleotide base modifications (for a review see Usman and Cedergren, 1992 TIBS 17, 34; Usman et al., 1994 Nucleic Acids Symp. Ser. 31, 163; Burgin et al., 1996 Biochemistry 35, 14090). Sugar modification of enzymatic nucleic acid molecules have been extensively described in the art (see Eckstein et al., International Publication PCT No. WO 92/07065; Perrault et al. Nature 1990, 344, 565-568; Pieken et al. Science 1991, 253, 314-317; Usman and Cedergren, Trends in Biochem. Sci. 1992, 17, 334-339; Usman et al. International Publication PCT No. WO 93/15187; Sproat, U.S. Pat. No. 5,334,711 and Beigelman et al., 1995 J. Biol. Chem. 270, 25702; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid catalysts of the instant invention.

Nucleic acid catalysts having chemical modifications which maintain or enhance enzymatic activity are provided. Such nucleic acid is also generally more resistant to nucleases than unmodified nucleic acid. Thus, in a cell and/or in vivo the activity may not be significantly lowered. As exemplified herein such ribozymes are useful in a cell and/or in vivo even if activity over all is reduced 10 fold (Burgin et al., 1996, Biochemistry, 35, 14090). Such ribozymes herein are said to “maintain” the enzymatic activity on all RNA ribozyme.

Therapeutic ribozymes delivered exogenously must optimally be stable within cells until translation of the target RNA has been inhibited long enough to reduce the levels of the undesirable protein. This period of time varies between hours to days depending upon the disease state. Clearly, ribozymes must be resistant to nucleases in order to function as effective intracellular therapeutic agents. Improvements in the chemical synthesis of RNA (Wincott et al., 1995 Nucleic Acids Res. 23, 2677; incorporated by reference herein) have expanded the ability to modify ribozymes by introducing nucleotide modifications to enhance their nuclease stability as described above.

A “nucleotide” as used herein is as recognized in the art to include natural bases (standard), and modified bases well known in the art. Such bases are generally located at the 1′ position of a sugar moiety. Nucleotide generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; all hereby incorporated by reference herein). There are several examples of modified nucleic acid bases known in the art and has recently been summarized by Limbach et al., 1994, Nucleic Acids Res. 22, 2183. Some of the non-limiting examples of base modifications that can be introduced into enzymatic nucleic acids without significantly effecting their catalytic activity include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine) and others (Burgin et al., 1996, Biochemistry, 35, 14090). By “modified bases” in this aspect is meant nucleotide bases other than adenine, guanine, cytosine and uracil at 1′ position or their equivalents; such bases may be used within the catalytic core of the enzyme and/or in the substrate-binding regions.

By “unmodified nucleoside” is meant one of the bases adenine, cytosine, guanine, uracil joined to the 1′ carbon of b-D-ribo-furanose.

By “modified nucleoside” is meant any nucleotide base which contains a modification in the chemical structure of an unmodified nucleotide base, sugar and/or phosphate.

Various modifications to ribozyme structure can be made to enhance the utility of ribozymes. Such modifications will enhance shelf-life, half-life in vitro, stability, and ease of introduction of such ribozymes to the target site, e.g., to enhance penetration of cellular membranes, and confer the ability to recognize and bind to targeted cells.

Administration of Trans-Splicing Nucleic Acid Molecules:

Sullivan et al., PCT WO 94/02595, describes the general methods for delivery of nucleic acid molecules. For example, ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Sullivan et al., supra and Draper et al., PCT WO93/23569 which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation to reach a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can localize the drug, for example, in certain tissue types, such as the tissues of the reticular endothelial system (RES). A liposome formulation which can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as the cancer cells.

The invention also features the use of the a composition comprising surface-modified liposomes containing poly (ethylene glycol) lipids (PEG-modified, or long-circulating liposomes or stealth liposomes). These formulations offer an method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwataet al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of these are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. Id. at 1449. These include sodium benzoate, sorbic acid and esters of p-hydroxybenzoic acid. In addition, antioxidants and suspending agents may be used. Id.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

Alternatively, the trans-splicing nucleic acid molecules of the instant invention can be expressed within cells from eukaryotic promoters (e.g., Izant and Weintraub, 1985 Science 229, 345; McGarry and Lindquist, 1986 Proc. Natl. Acad. Sci. USA 83, 399; Scanlon et al., 1991, Proc. Natl. Acad. Sci USA, 88, 10591-5; Kashani-Sabet et al., 1992 Antisense Res. Dev., 2, 3-15; Dropulic et al., 1992 J. Virol, 66, 1432-41; Weerasinghe et al., 1991 J. Virol, 65, 5531-4; Ojwang et al., 1992 Proc. Natl. Acad. Sci. USA 89, 10802-6; Chen et al., 1992 Nucleic Acids Res., 20, 4581-9; Sarver et al., 1990 Science 247, 1222-1225; Thompson et al., 1995 Nucleic Acids Res. 23, 2259; Good et al., 1997, Gene Therapy, 4, 45; all of the references are hereby incorporated in their totality by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992 Nucleic Acids Symp. Ser., 27, 15-6; Taira et al., 1991, Nucleic Acids Res., 19, 5125-30; Ventura et al., 1993 Nucleic Acids Res., 21, 3249-55; Chowrira et al., 1994 J. Biol. Chem. 269, 25856; all of the references are hereby incorporated in their totality by reference herein).

Trans-splicing nucleic acid molecules that cleave target molecules are expressed from transcription units (see for example Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the ribozymes are delivered as described above, and persist in target cells. Alternatively, viral vectors may be used that provide for transient expression of ribozymes. Such vectors might be repeatedly administered as necessary. Once expressed, the ribozymes cleave the target MRNA. The active ribozyme contains an enzymatic center or core equivalent to those in the examples, and binding arms able to bind target nucleic acid molecules such that cleavage at the target site occurs. Other sequences may be present which do not interfere with such cleavage. Delivery of ribozyme expressing vectors could be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12,510).

An expression vector comprising nucleic acid sequence encoding at least one of the trans-splicing nucleic acid molecules, such as a ribozyme, of the instant invention is disclosed. The nucleic acid sequence encoding the nucleic acid catalyst of the instant invention is operable linked in a manner which allows expression of that nucleic acid molecule.

Also, featured is an expression vector compriseing: a transcription initiation region (e.g., eukaryotic pol I, II or III initiation region); b) a transcription termination region (e.g., eukaryotic pol I, II or III termination region); c) a gene encoding at least one of the nucleic acid catalyst of the instant invention; and wherein said gene is operably linked to said initiation region and said termination region, in a manner which allows expression and/or delivery of said nucleic acid molecule. The vector may optionally include an open reading frame (ORF) for a protein operably linked on the 5′ side or the 3′-side of the gene encoding the nucleic acid catalyst of the invention; and/or an intron (intervening sequences).

Transcription of the trans-splicing construct sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990 Proc. Natl. Acad. Sci. USA, 87, 6743-7; Gao and Huang 1993 Nucleic Acids Res., 21, 2867-72; Lieber et al., 1993 Methods Enzymol., 217, 47-66; Zhou et al., 1990 Mol. Cell. Biol., 10, 4529-37). Several investigators have demonstrated that ribozymes expressed from such promoters can function in mammalian cells (e.g. Kashani-Sabet et al., 1992 Antisense Res. Dev., 2, 3-15; Ojwang et al., 1992 Proc. Natl. Acad. Sci. USA, 89, 10802-6; Chen et al., 1992 Nucleic Acids Res., 20, 4581-9; Yu et al., 1993 Proc. Natl. Acad. Sci. USA, 90, 6340-4; L'Huillier et al., 1992 EMBO J. 11, 4411-8; Lisziewicz et al., 1993 Proc. Natl. Acad. Sci. U.S.A., 90, 8000-4; Thompson et al., 1995 Nucleic Acids Res. 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther. 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

Applicant also discloses an expression vector comprising nucleic acid sequence encoding at least one of the nucleic acid molecule of the invention, in a manner which allows expression of that nucleic acid molecule. The expression vector comprises in one embodiment; a) a transcription initiation region; b) a transcription termination region; c) a gene encoding at least one said nucleic acid molecule; and wherein said gene is operably linked to said initiation region and said termination region, in a manner which allows expression and/or delivery of said nucleic acid molecule. In another preferred embodiment the expression vector comprises: a) a transcription initiation region; b) a transcription termination region; c) an open reading frame; d) a gene encoding at least one said nucleic acid molecule, wherein said gene is operably linked to the 3′-end of said open reading frame; and wherein said gene is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a gene encoding at least one said nucleic acid molecule; and wherein said gene is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a gene encoding at least one said nucleic acid molecule, wherein said gene is operably linked to the 3′-end of said open reading frame; and wherein said gene is operably linked to said initiation region, said intron, said open reading frame and said termination region, in a manner which allows expression and/or delivery of said nucleic acid molecule.

The following is an example of various ribozyme constructs used to show the operability of the claimed invention. Those in the art will recognize that this example indicates the utility of the invention for both in vitro and in vivo splicing reactions. While significant utility will be attained in vivo by use of the present invention, those in the art will also recognize that in vitro utility is important and can be used to create chimeric transcripts for use in laboratory situations or in a clinical setting.

### EXAMPLES

### Example 1

### Mapping Accessible Regions on the Beta-Globin Transcript

Methods:

Generation of RBC precursors from normal umbilical cord blood and from peripheral blood of sickle cell patients. Umbilical cord blood samples were obtained from labor and delivery and peripheral blood samples were obtained from sickle cell patients with hemoglobin SC disease undergoing scheduled phlebotomy. Mononuclear cells were isolated by ficol-hypaque gradient separation and resuspended at 1×10⁶ cells/ml in BIT 9500 serum free media (Stem Cells Technology), supplemented with Flt-3 ligand (25 ng/ml, Immunex), IL-3 (2.5 ng/ml, R&D Inc.), and Erythropoeitin (1 u/ml, R&D Inc.). These cells were then cultured at 37° C. overnight and transferred to fresh plates to eliminate adherent cells.

Transfection of RBC precursors. RBC precursors (1×10⁶) were resuspended in Opti-MEM (200 ml, Gibco-BRL), and ribozymes (2.5-5 mg) were lipofected into these cells using DMRIE-C (20 ml, Gibco-BRL) in Iml Opti-MEM for four hours. Then, DMEM (Gibco-BRL) with 10% fetal calf serum (1 ml) and erythropoeitin (2 u/ml) were added to the cells. Total RNA was isolated using TRI Reagent (Molecular Research Center) 16-24 hours after transfection. Transfection of these cells with a reporter RNA demonstrated that 1-2% of the erythrocyte precursors take up RNA.

Generation of the mapping library. The mapping library was generated by PCR amplification of the plasmid pT7L-21 with a 5′ primer containing a randomized sequence at the positions corresponding to the ribozyme's IGS (5′-GGGGGGATCCTAATACGACTCACTATAGNNNNNAAAAGTTATCA

GGCATGCACC) and a 3′ primer specific for3′ exon tag sequences present in the pT7L-21 plasmid

(5′-AGTAGTCTTACTGCAGGGGCCTCTTCGCTATTACG). The resulting cDNA library was in vitro transcribed using T7 RNA polymerase to generate the RNA mapping library.

Trans-splicing reactions. Ribozyme-3′ exon (100-500 μM) and substrate RNAs (1-5 mM or 1 μg cellular RNA) were denatured at 95° C. for 1 min in reaction buffer (50 mM HEPES pH7.0, 150 mM NaCl and 5 mM MgCl₂) and then equilibrated at 37° C. for 3 min. The substrates were then added to the ribozymes along with guanosine (100 mM) to start the reactions, which proceeded at 37° C. for 3 hours. For reactions containing radiolabeled ribozyme, aliquots were removed at the times indicated and added to an equal volume of EDTA (10 mM) to stop the reaction. Reaction products were analyzed on a 4% polyacrylamide gel containing urea (8M).

RT-PCR analysis. Trans-splicing products were reverse-transcribed at 37° C. for 20 minutes in the presence of L-argininamide (10 mM) from a primer specific for the 3′ exon sequence as previously described. The resulting cDNAs were amplified for 30 cycles (in vitro ribozyme reactions) or 30-90 cycles (in vivo ribozyme reactions) using a 3′ exon primer (3′ tag primer: 5′-ATGCCTGCAGGTCGACTC, 3′gamma-globin primer: 5′-CCGGAATTCCCTTGTCCTCCTCTGTGA) and a 5′ primer specific for the beta-globin mRNA (5′GGGGATCCCTGTGTTCACTAGCAACC). The amplified products were separated on a 3% agarose gel and visualized by ethidium bromide staining.

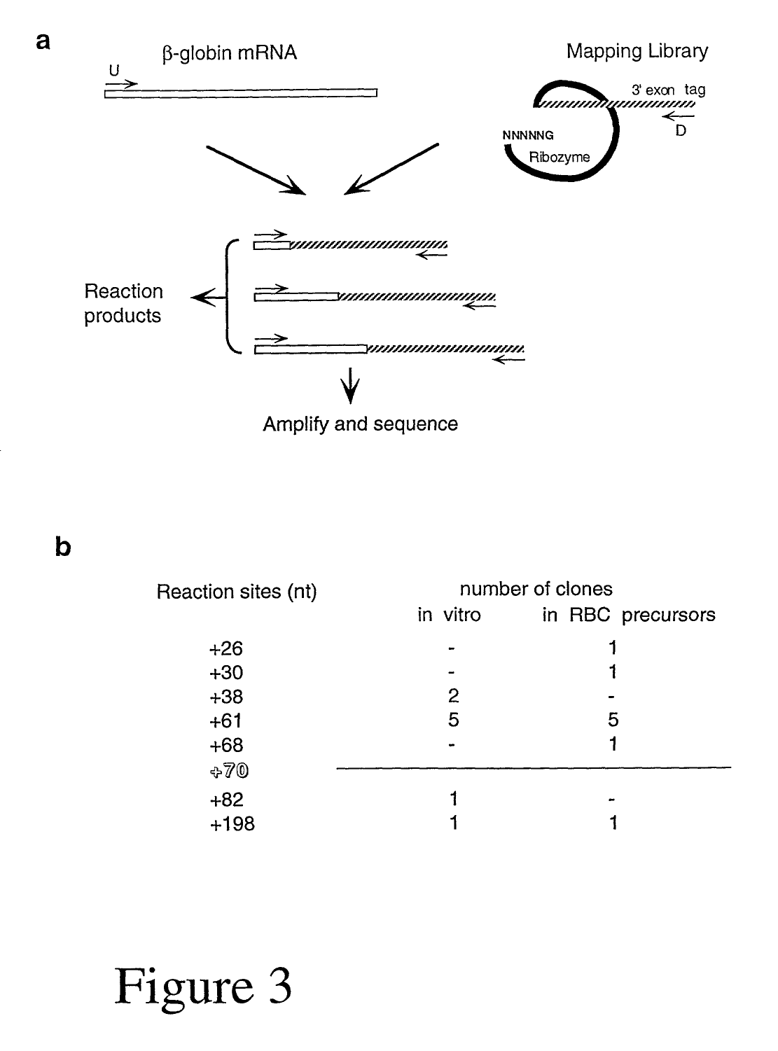

To ascertain which regions of the beta-globin transcript are accessible to ribozymes, we developed a novel RNA mapping strategy that employs a trans-splicing ribozyme library and RNA tagging. To generate the mapping library, the guide sequence of the Tetrahymena group I trans-splicing ribozyme was randomized such that the 5′ end of the RNAs in the library begin with 5′-GNNNNN-3′ where “G” represents guanine and “N” represents equal amounts of the 4 nucleotides¹⁵ (FIG. 3a). To map the beta-globin transcript in vitro, the mapping library was incubated with total RNA isolated from erythrocyte precursors under splicing conditions. To identify accessible uridine residues, the trans-splicing reaction products were reverse transcribed (RT) and amplified by the polymerase chain reaction (PCR) using primers specific for the ribozyme's 3′ exon tag⁶ and for the beta-globin target RNA (FIG. 3a). The resulting cDNAs were then sequenced to determine which uridine residues were present at the ribozyme reaction sites. From such analysis, the uridine at position 61 of beta-globin RNA appears particularly accessible because 5 out of 9 sequenced clones contain splice junctions at this nucleotide (FIG. 3b). To determine which nucleotides are accessible on beta-globin transcripts inside cells, the mapping library was transfected into erythrocyte precursors. Total RNA was isolated from these cells and reactive uridines identified by RT-PCR amplification and sequence analysis. The uridine at position 61 also appears to be particularly accessible in vivo because in 5 of the 9 clones examined the 3′ exon tag had been spliced onto this nucleotide (FIG. 3b). These mapping results, taken together with the fact that sickle beta-globin transcripts contain a point mutation at position 70, encouraged applicant to focus on developing ribozymes that recognize the uridine present at position 61 on the beta-globin MRNA. Thus, the internal guide sequence on the L-21 trans-splicing ribozyme was changed to 5′-GGGUGC-3′ to generate a ribozyme, called Rib61, specific for site 61. In addition, an inactive version of this ribozyme, called Rib61d, which lacks part of the catalytic core of the enzyme was generated to control for the importance of ribozyme activity in these studies.

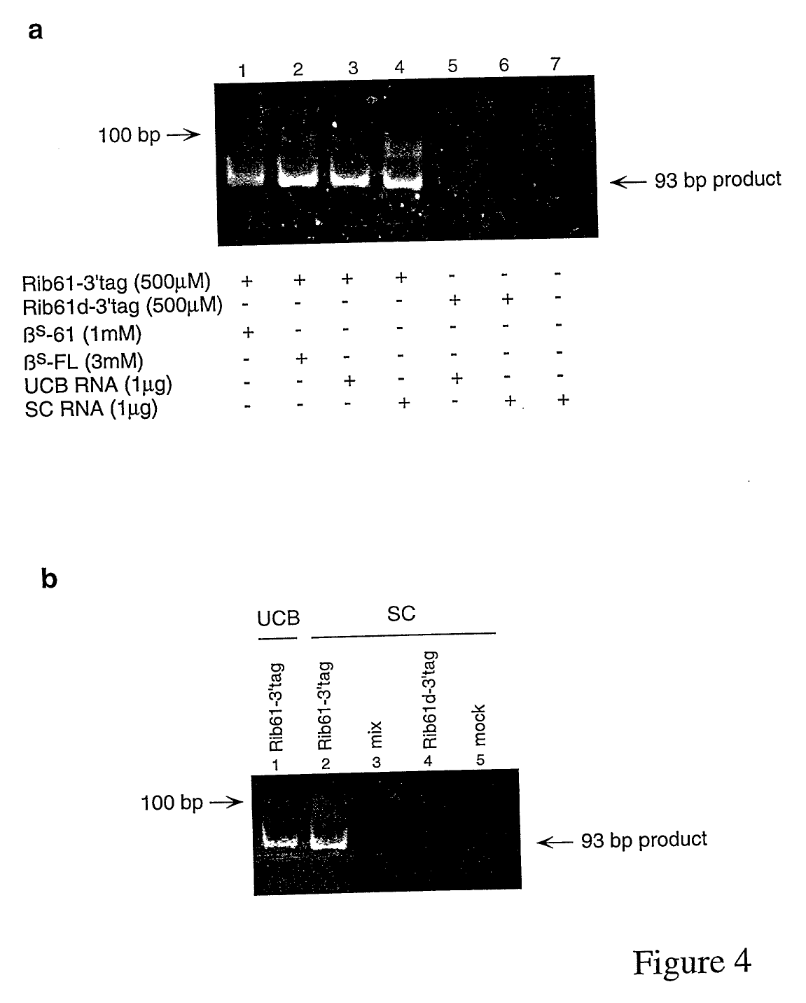

Rib61 can trans-splice a3′ exon tag onto beta-globin transcripts in vitro and in erythrocyte precursors (FIG. 4). The trans-splicing ribozymes, Rib61-3′tag and Rib61d-3′tag, were incubated under splicing conditions with β^{S}-globin RNA generated by in vitro transcription or total RNA isolated from erythrocyte precursors. To determine if trans-splicing had occurred in any of the RNA samples, RT-PCR analyses were performed using one primer specific for the beta-globin target RNA and the other primer specific for the3′ exon tag sequence (FIG. 4a). An amplified fragment of the expected size (93 base pairs) was generated from samples containing Rib61-3′tag and either in vitro transcribed β^{S}-globin RNA or total RNA isolated from sickle cell patient and UCB derived RBC precursors. No such RT-PCR product was generated from samples that lack a ribozyme or that contain the inactive version of the ribozyme.

### Example 2

### Repair of Sickle Beta-Globin Transcripts

Sickle cell anemia is the most common heritable hematological disease yet no curative treatment exists for this disorder. Moreover the intricacies of globin gene expression have made the development of gene therapy based treatments for hemaglobinopathies difficult. Applicant describes an alternative genetic approach to sickle cell therapy. A trans-splicing group I ribozyme can be employed to amend mutant beta-globin transcripts in erythroid lineage cells. To determine which regions of the beta-globin transcript are accessible to ribozymes inside cells, a novel RNA mapping strategy was developed that employs a trans-splicing ribozyme library and RNA tagging From such analysis, the uridine at position 61 of beta-globin RNA appears particularly accessible. A trans-splicing ribozyme that recognizes this nucleotide reacts with beta-globin transcripts with high fidelity in erythrocyte precursors derived from normal umbilical cord blood or peripheral blood from individuals with sickle cell disease. Moreover such splicing can convert sickle beta -globin transcripts into RNAs encoding the anti-sickling protein gamma-globin. These results suggest that trans-splicing ribozymes may represent a new approach to the treatment of sickle cell and other genetic diseases.

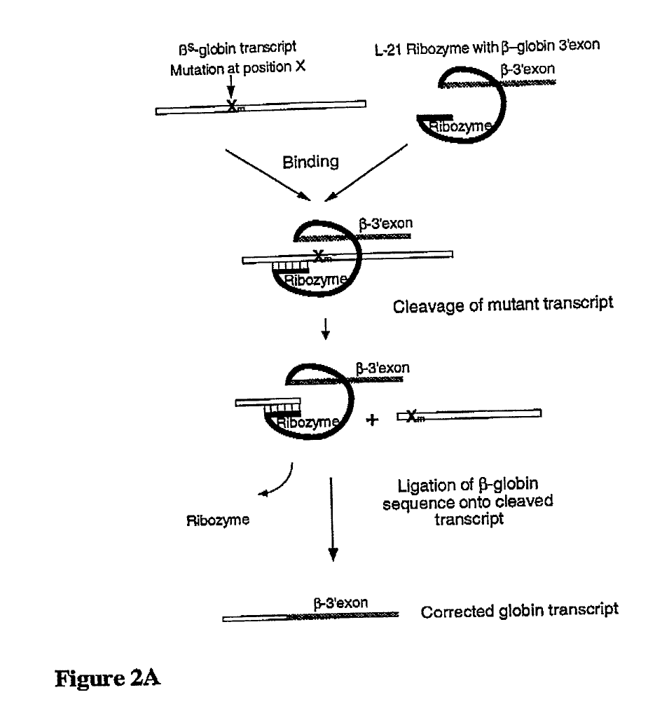

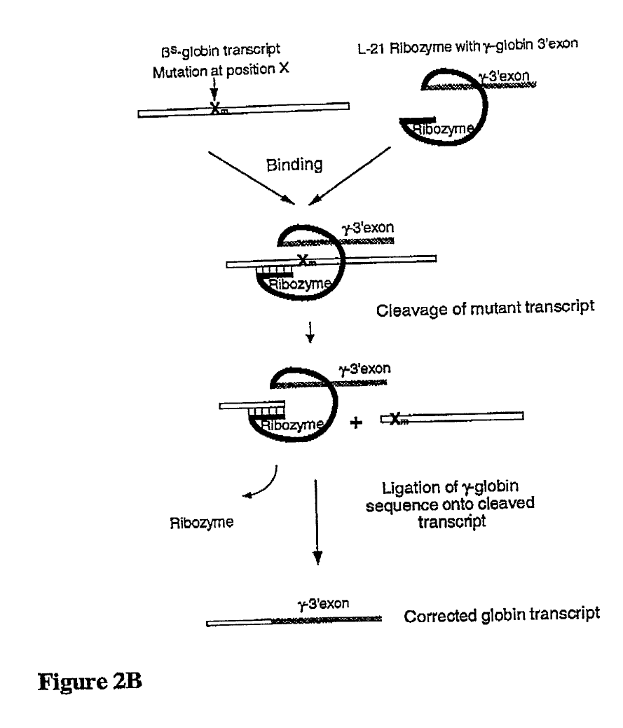

To determine if trans-splicing could be employed to repair mutant transcripts associated with a common genetic disorder, applicant tested whether such splicing ribozymes could amend mutant P-globin transcripts in erythroid lineage cells derived from the peripheral blood of patients with sickle cell disease (FIG. 2A). More specifically, we wanted to determine if splicing ribozymes could convert sickle β-globin (β^{S}-globin) transcripts into RNAs encoding g-globin because fetal hemoglobin (Hb F) which contains γ-globin has been shown to greatly impede polymerization of hemoglobin S (Hb S). In this splicing reaction, the ribozyme recognizes the sickle beta-globin transcript by base pairing to an accessible region of the RNA upstream of the mutant nucleotide via an internal guide sequence (IGS), cleaves the β^{S}-globin RNA, releases the mutation containing cleavage product and splices on the revised sequence for the globin transcript (FIG. 2A).

To determine if trans-splicing ribozymes can react with beta-globin transcripts in clinically relevant cells, we generated erythrocyte precursors from normal umbilical cord blood (UCB) and from peripheral blood from patients with sickle cell disease by culturing the blood cells in serum free conditions supplemented with erythropoietin, Flt-3 ligand and IL-3. Nucleated red blood cells (RBC) appear by day 7 under these culture conditions and by three weeks they constitute 70-90% of the total number of cells in the culture as evidenced by Wright-Giemsa and immunofluorescent staining (FIG. 2). Moreover because the majority of these erythroid lineage cells are late RBC precursors (FIG. 2), they are rich in globin transcripts making them ideal for the RNA repair studies described herein.

To determine if Rib61-3′tag could react with beta-globin transcripts inside primary human cells, the ribozyme was transfected into erythrocyte precursors derived from UCB and sickle cell patients. Total RNA was isolated from these cells and analyzed via RT-PCR to determine if trans-splicing products were present in any of the cellular samples (FIG. 4b). An amplified fragment of the expected size (93 base pairs) was generated from the RNA samples isolated from sickle cell patient and UCB derived RBC precursors that had been transfected with the active ribozyme. By contrast no such product was generated from RNA samples isolated from cells that were not transfected or were transfected with the inactive ribozyme. To determine if transsplicing was occurring during RNA isolation and analysis, Rib61-3′Tag was added to the RNA extraction buffer used to isolate total RNA from a sample of mock transfected erythrocyte precursors. No amplification product was generated when this “mixed” RNA sample was analyzed by RT-PCR (FIG. 4b) suggesting that the observed trans-splicing products were generated inside the RBC precursors and not during RNA analysis.

Trans-splicing nucleic acid molecules can be employed to correct a broad array of mutant transcripts associated with a variety of genetic disorders. Here we demonstrate that a ribozyme can amend a disease related transcript, mutant β-globin mRNA, in clinically relevant cells, erythrocyte precursors derived from sickle cell patients. RNA repair may be a particularly appropriate genetic approach with which to treat sickle cell disease because the process should restore the regulated expression of anti-sickling versions of β-globin and simultaneously reduce the production of β^{S}-globin (FIG. 2). Moreover, the efficiency of β-globin RNA repair will likely not have to be 100% to benefit patients. Sickle cell trait is a benign condition that is not associated with increased morbidity or mortality and sickle cell patients that express γ-globin at 10-20% the level of β^{S}-globin in the majority of their RBCs have greatly improved clinical prognoses. The results presented here suggest that ribozyme-mediated repair of mutant RNAs may prove to be a useful approach to treat sickle cell disease and other inherited disorders.

The trans-splicing reaction to repair mutant beta-globin transcript can be tested in vivo using a variety of sickle cell disease animal models, prior to testing in humans (for e.g., see Ryan et al., 1997, Science 278, 873; Paszty et al., 1997, Science. 278,876; both are incorporated by reference herein).

### Example 3

### Trans-Splicing γ-Globin RNA.

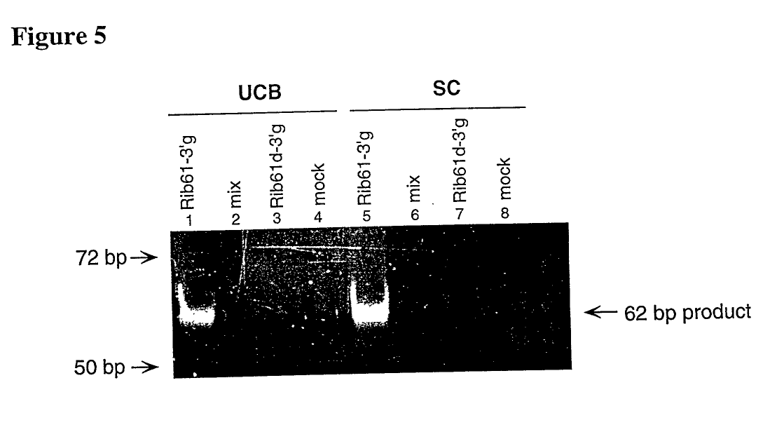

To create a trans-splicing ribozyme that could convert β^{S}-globin transcripts into RNAs encoding γ-globin (FIG. 2B), the3′ exon on Rib61-3′tag was changed to contain the human γ-globin cDNA sequence (nucleotides 29-545). This trans-splicing ribozyme, called Rib61-3′γ, can quickly and accurately trans-splice its 3′γ-globin exon onto β^{S}-globin RNAs in vitro. Radiolabeled Rib61-3′γwas allowed to react with an excess of unlabeled full length (β^{S}-FL) or truncated (β^{S}-61) β^{S}-globin substrate RNA (FIG. 5). In both reactions, Rib61-3′γ was quickly converted to free ribozyme (Rib) plus ligated globin exons (β^{S}-61-3′γ) with an approximate half-time (t_{½}) of 60 minutes. Rib61-3′γ reacted even faster (t_{½}˜25 minutes) with a short 13 nucleotide substrate (5′SA₅). The inactive version of the ribozyme (Rib61d-3′γ) was unable to mediate this splicing reaction (data not shown).

To determine if this trans-splicing ribozyme could convert “authentic” β^{S}-globin transcripts into RNAs encoding γ-globin, we transfected Rib61-3′γ into erythirocyte precursors derived from sickle cell patients and UCB. Total RNA was harvested from these cells and RT-PCR analyses were performed to determine if amended β-globin transcripts were present in the RNA samples (FIG. 5). An amplified fragment of the expected size (62 base pairs) was generated from RNA samples isolated from erythrocyte precursors derived from sickle cell patients or UCB that had been transfected with the active ribozyme, Rib61-3′. No such product was generated from RNA samples isolated from cells that were not transfected or were transfected with the inactive ribozyme. Moreover, no amplification product was generated from RNA samples in which Rib61-3′ was added to the RNA extraction buffer prior to lysing the mock transfected cells (FIG. 5). Thus the amended globin RNAs were almost assuredly generated by the trans-splicing ribozyme inside the RBC precursors and not during RNA analysis.

To confirm that the amplified DNA products were generated from globin transcripts that had been correctly spliced, we subcloned and sequenced the amplified fragments. Sequence analysis of eight different subclones derived from sickle cell patient samples demonstrated that in each case the ribozyme had correctly spliced its γ-globin 3′exon onto nucleotide 61 of the β-globin target transcript and in the process maintained the open reading frame for the translation of the MRNA. Thus, trans-splicing ribozymes appear to be able to revise mutant globin transcripts in primary human RBC precursors with high fidelity.

Uses

Gene mapping and human genome sequencing provides the genetic basis for an increasing number of inherited diseases. With each discovery or identification of a new disease-related gene there is an opportunity to develop gene therapy based treatments.

Trans-splicing nucleic acid molecules can be used to correct the defective transcripts issuing from mutant genes. This approach will be valuable for the treatment of the many genetic diseases caused by a common set of specific mutations which do not affect the expression of the mutant gene. For example, the genetic basis of many globin diseases is well understood. Targeted trans-splicing can repair or correct globin transcripts that are either truncated or contain point mutations. In the process, the cellular expression pattern of these genes is maintained. Therefore, targeted trans-splicing represents an important, novel strategy for the treatment of many genetic diseases.

As noted above, trans-splicing may also be accomplished without the use of ribozymes. It has been demonstrated that spliced leader sequences from lower eucaryotes can be trans-spliced onto mammalian3′ splice sites in tissue culture cells (Bruzik et al., Nature 360, 692 (1992)). Trans-splicing in this case is mediated by the spliceosome or splicing factors. There are several reports of protein dependent trans-splicing reactions in a variety of systems (see for example Ghetti et al., 1995, Proc. Natl. Acad. Sci., 92, 11461; Bruzik et al., supra). Thus, it is possible to employ spliceosomes to alter the sequence of targeted transcripts for some desired end via targeted trans-splicing.

Trans-splicing nucleic acid molecules can be used to attach any Tag to a target nucleic acid molecules. The molecular Tags do not have to be composed of nucleic acid sequence. One can simply alter the molecular composition of the Tag attached to the ribozyme. Just as in vitro selection has allowed for the generation of novel ribozymes with new activities, similar selection should allow for the development of ribozymes that can covalently attach novel Tags to target molecules. Thus ribozymes can be developed that can covalently modify a range of target molecules in a variety of ways. Such ribozymes can be used for a number of diagnostics and in manufacturing applications. For example, if one wants to a make a soap that has a specific type of chemical linkage that is difficult to generate by classical organic chemistry techniques, now one can consider the generation of a ribozyme that will recognize the precursor of the final soap product, say a certain lipid, catalytically react with the lipid and covalently transfer a molecular group (the Tag in this case) to the target lipid to modify it in the desired manner. Thus, the ability of ribozymes to covalently attach molecules to specific substrate molecules may allows us to employ ribozyme in ways that were not previously envisioned. In another example, Tags such as biotin can be attached to a target nucleic acid molecule (e.g., a pathogenic virus RNA)in vitro using trans-splicing ribozymes in biological sample from a patient. The extent of biotin attachment to the target RNA can be used as a measure of viral load in the patient; such measurements can be made using standard techniques such as using avidin to isolate biotin tagged RNA from the sample and quantifying the biotin tagged RNA.

Other embodiments are within the following claims.

1. A method of replacing a region of a mutant beta-globin RNA molecule containing at least one mutation, with a desired beta-globin sequence using a trans-splicing nucleic acid molecule to generate a beta-globin transcript that can yield protein product that would exhibit normal beta-globin attribute, comprising the steps of:
a) contacting the mutant beta-globin RNA molecule with the trans-splicing nucleic acid molecule, wherein said trans-splicing molecule comprises the desired beta-globin sequence; and
b) said contacting in section (a) is carried out under conditions suitable for a trans-splicing reaction to occur, wherein the mutant region of the mutant beta-globin RNA is replaced.

2. A method of converting a mutant beta-globin RNA molecule containing at least one mutation, into a chimeric beta-gamma-globin sequence using a trans-splicing nucleic acid molecule to generate a transcript that can yield protein product that would exhibit normal gamma-globin attribute, comprising the steps of:
a) contacting the mutant beta-globin RNA molecule with the trans-splicing nucleic acid molecule, wherein said trans-splicing molecule comprises the gamma-globin sequence; and
b) said contacting in section (a) is carried out under conditions suitable for a trans-splicing reaction to occur, wherein the mutant beta-globin RNA is converted.

3. A method of attaching a Tag moiety other than nucleic acid to a target nucleic acid using enzymatic trans-splicing nucleic acid molecules, comprising the step of contacting the target nucleic acid molecule with the enzymatic trans-splicing nucleic acid molecule comprising a Tag, under conditions suitable for the attachment of the Tag.

4. A method of identifying at least one region in a target RNA molecule that is accessible to interaction with a separate nucleic acid molecule comprising the steps of:
a) contacting the target RNA molecule with an enzymatic nucleic acid molecule with trans-splicing activity, wherein the enzymatic nucleic acid comprises a Tag sequence;
b) said contacting in section (a) is carried out under conditions suitable for trans-splicing reaction to occur, wherein the Tag sequence is covalently attached to the target RNA molecule to form a chimeric RNA molecule; and
c) identifying the accessible region in the target RNA by identifying the region in the target RNA molecule where the Tag sequence has been inserted.

5. The method of any of claims 1 or 2, wherein said trans-splicing nucleic acid molecule is an enzymatic nucleic acid molecule.

6. The method of claim 5, wherein said enzymatic nucleic acid molecule is derived from a group I intron.

7. The method of claim 5, wherein said enzymatic nucleic acid molecule is derived from a group II intron.

8. The method of claim 4, wherein said enzymatic nucleic acid molecule is derived from a group I intron.

9. The method of claim 4, wherein said enzymatic nucleic acid molecule is derived from a group II intron.

10. The method of claim 1 or 2, wherein said trans-splicing occurs in the presence of one or more splicing factors.

11. The method of any of claims 1-4, wherein said contacting comprises providing a vector encoding said trans-splicing nucleic acid molecule comprising said separate nucleic acid molecule.

12. The method of claim 4, wherein said separate nucleic acid molecule is an enzymatic nucleic acid molecule.

13. The method of claim 4, wherein said separate nucleic acid molecule is an antisense nucleic acid molecule.

14. The method of claim 3, wherein said Tag moiety is selected from a group consisiting of a lipid, carbohydrate, vitamin, biotin, a fluorescence compound, peptide, aminoacid, and an antibiotic.

15. The method of claim 4, wherein said enzymatic nucleic acid comprises a substrate binding region, wherein the substrate binding region comprises a randomized region.
